(19) United States
(12) Patent Application Publication
(10) Pub. No.: US 2024/0319200 A1
(43) Pub. Date: Sep. 26, 2024

(54) Covalent Molecular Glue Stabilizers and Platform

(71) Applicant: The Regents of the University of California, Oakland, CA (US)

(72) Inventors: Daniel K. Nomura, Berkeley, CA (US); Qian Shao, Berkeley, CA (US)

(73) Assignee: The Regents of the University of California, Oakland, CA (US)

(21) Appl. No.: 18/736,528

(22) Filed: Jun. 7, 2024

Related U.S. Application Data
(63) Continuation of application No. PCT/US22/51591, filed on Dec. 1, 2022.
(60) Provisional application No. 63/295,528, filed on Dec. 31, 2021.
(60) Provisional application No. 63/295,529, filed on Dec. 31, 2021.

(51) Int. Cl. G01N 33/58; A61K 31/407; A61K 31/4439; C07D 487/10

(52) U.S. Cl. CPC G01N 33/582; A61K 31/407; A61K 31/4439; C07D 487/10; G01N 2500/02

(57) ABSTRACT

A novel drug discovery platform identifies compounds for therapeutically targeting 14-3-3 proteins and sequestering and inhibiting the activities of various disease-relevant proteins. Therapeutic compositions comprise a scaffold for therapeutically targeting 14-3-3 proteins and sequestering and inhibiting the activities of various disease-relevant proteins.

### CROSS-REFERENCES TO RELATED APPLICATIONS

This application is a continuation of PCT/US22/51591, filed Dec. 1, 2022, which claims priority to U.S. Provisional Application Nos. 63/295,528 and 63/295,529, both filed Dec. 31, 2021, the disclosures of which are hereby incorporated by reference in its entirety for all purposes.

### INTRODUCTION

One of the challenges facing cancer drug discovery is that >85% of the proteome is currently considered “undruggable” because most proteins do not possess a well-defined binding pocket or “ligandable hotspot” that can be pharmacologically and functionally targeted for therapeutic benefit¹. Tackling the undruggable proteome not only requires the development of innovative technologies for discovering proteome-wide ligandable hotspots and corresponding ligands, but also necessitates the invention of new therapeutic modalities that enable the functional manipulation of undruggable targets to confer therapeutic benefit. Induced proximity-based therapeutic modalities (IPTMs) have arisen as a powerful therapeutic strategy for tackling undruggable protein targets by using bifunctional or monovalent small-molecules to induce the proximity of a target protein with a modulator protein to functionally manipulate target proteins to confer therapeutic benefit. An example of a very popular IPTM is Proteolysis Targeting Chimeras (PROTACs) that use heterobifunctional small-molecules to induce the proximity of E3 ligases with target proteins to ubiquitinate and eliminate the protein through proteasome-mediated degradation². However, inducing the proximity of proteins to degrade them is just the tip of the iceberg of IPTMs that can exploited for therapeutic benefit. These PROTACs also possess higher molecular weights that pose a challenge for drug-like properties. Furthermore, heterobifunctional IPTMs require a protein-targeting ligand that binds to the target protein with decent affinity, which may not be possible for many undruggable proteins that do not possess deep enough binding pockets.

Molecular glues, monovalent small-molecules that induce the proximity of proteins to confer neomorphic protein functions, are very attractive therapeutic paradigms that overcome many of the challenges involved with targeting undruggable proteins or with PROTACs. Molecular glues, through their ability to form ternary complexes, can target and bring together two shallow protein interfaces that alone may not be targetable with a small-molecule. Molecular glues are also monovalent molecules that are usually much smaller in molecular weight and more drug-like. Several molecular glues have led to game-changing drugs. These include natural products like rapamycin that glue together FKBP12 with mTORC1 to partially disrupt mTORC1 function or fully synthetic small-molecules such as pomalidomide that induce the proximity of the E3 ligase cereblon with neo-substrate transcription factors such as Ikaros to induce their ubiquitination and degradation³⁻⁵. However, most molecular glues have been discovered fortuitously and there have not been good target-based systematic approaches for discovering new molecular glues to tackle undruggable targets in cancer in a rational manner. One approach to disrupt the function of undruggable cancer targets, such as nuclear oncogenic transcription factors (e.g. MYC, CTNNB1, MYCN, FOS, JUN, etc) or signaling proteins (e.g. oncogenic kinases) is to sequester these proteins outside of the nucleus or away from their endogenous protein interactions to inhibit their function.

14-3-3 proteins are a family of proteins that naturally bind and sequester hundreds of transcription factors, kinases, and receptors when they are in their phosphorylated state⁶. There have been substantial efforts to develop molecular glues that enhance 14-3-3 interactions with oncogenic transcription factors and kinases using a fortuitously discovered natural product fusicoccin A and its analogs to sequester and inhibit the functions of these oncogenes for cancer therapy. However, efforts to diversify upon this natural product scaffold to glue together 14-3-3 with transcription factors beyond already-druggable ones such as estrogen receptor (ERα) have not been successful. Furthermore, working with natural product scaffolds is very synthetically challenging. Discovering a fully synthetic and covalent 14-3-3 molecular glue scaffold that can broadly enhance interactions between 14-3-3 and many oncogenic proteins to inhibit their function by exploiting the reactivity and the ability of covalent ligands to access cryptic and shallow pockets would substantially enable efforts to sequester and inhibit a broad range of hitherto undruggable oncogenic targets in cancer.

Relevant Literature includes
Sijbesma, ACS Med. Chem. Lett. 2021, 12, 6, 976-982; Exploration of a 14-3-3 PPI Pocket by Covalent Fragments as Stabilizers; https://www.ncbi.nlm.nih.gov/pmc/articles/PMC8201753/
Falcicchio Chem Sci. 2021 Oct. 13; 12(39): 12985-12992; Cooperative stabilisation of 14-3-3σ protein-protein interactions via covalent protein modification; https://www.ncbi.nlm.nih.gov/pmc/articles/PMC8513901/.

### SUMMARY OF THE INVENTION

We deployed novel covalent chemoproteomic platforms and developed novel 14-3-3-based covalent molecular glues that sequestered and inhibited oncogenic transcription factors, including estrogen receptor alpha (ERα) and hitherto undruggable oncogenic Hippo pathway transcription factors YAP1 and TAZ (WWTR1).

Our disclosure is the first to target the endogenous cysteine C38 of 14-3-3 with a drug-like chemical scaffold, not only with relatively druggable transcription factors such as the estrogen receptor (ERα), but also with oncogenic transcription factors such as YAP and TAZ. In contrast to compounds like 2-((3-aminopropyl)amino)ethanethiol (WR-1065), identifying new drug-like chemical scaffolds that comply with drug development criteria, like Lipinski's rule of five for drug-likeness, and/or with Ghose's filter, provide malleable frameworks for rational SAR development. Scaffolds that are capable of targeting the wild-type 14-3-3 (not engineered ones) that can stabilize interactions with proteins beyond ER-α has been a major challenge in developing 14-3-3 stabilizing drugs.

The invention provides a drug discovery platform for identifying compounds for therapeutic targeting 14-3-3 proteins and sequestering and inhibiting the activities of various disease-relevant proteins (e.g. oncogenic transcription factors that drive cancer and inhibiting them by sequestering them).

In an aspect the invention provides a method to identify a drug-like molecular glue by measuring fluorescence polarization of a 14-3-3 protein and a fluorescently-labeled phosphopeptide substrate thereof comprising transcription factor 14-3-3 interacting sequences, in the presence of candidate cysteine-reactive covalent ligands to identify a ligand that covalently reacts with Cys38 of the 14-3-3 protein and stabilizes or enhances interaction of the 14-3-3 protein with the substrate.

In embodiments:
the transcription factor is an oncogenic transcription factor such as YAP and TAZ;
the transcription factor is ERα;
the label is 5-FAM or rhodamine;
the 14-3-3 protein is wild-type (not engineered);
the 14-3-3 protein is stratifin (SFN);
the ligands or ligand has an n-octanol-water partition coefficient, K_{ow} (log P) in −0.4 to +5.6 range; and/or
the ligands or ligand complies with Lipinski's rule of five for drug-likeness, and/or with Ghose's filter (Ghose et al, 1999, J Comb Chem. 1 (1): 55-68): (a) Partition coefficient log P in −0.4 to +5.6 range; (b) Molar refractivity from 40 to 130; (c) Molecular weight from 180 to 480; and (d) Number of atoms from 20 to 70 (includes H-bond donors [e.g. OHs and NHs] and H-bond acceptors [e.g. Ns and Os]).

In an aspect the invention provides a drug discovery platform configured for a method herein.

In an aspect the invention provides a compound comprising a 7-substituted, 2,7-diazaspiro[4.4]nonane-2-acrylamide, or a pharmaceutically-acceptable salt thereof.

In embodiments:
the compound is 7-substituted with R, wherein R is selected from optionally substituted heteroatom and optionally substituted, optionally hetero-, optionally cyclic C1-C18 hydrocarbyl, wherein R may form a ring by covalently attaching to the C1 or C5 carbon of the proximate pyrrolidinyl;
the compound is 7-substituted with R, wherein R is methyl substituted with an optionally substituted, C3-C10 cyclic or heterocyclic group;
the compound is is 7-substituted with R, wherein R is methyl substituted with an optionally substituted, C5, C6 or C10 aryl or heteoraryl group comprising 1, 2 or 3 N, O or S heteroatoms;
the compound comprises a structure of Table 1; and/or
the compound covalently reacts with Cys38 of the 14-3-3 protein and stabilizes or enhances interaction of a 14-3-3 protein with a substrate thereof.

In an aspect the invention provides compounds that are structural and functional analogues of EN-171:

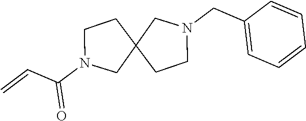 [SMILES: C=CC(=O)N1CCC2(CCN(Cc3ccccc3)C2)C1]

In an aspect the invention provides a compound of general formula I:

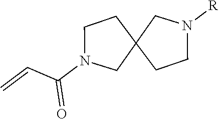

wherein R is optionally substituted heteroatom and optionally substituted, optionally hetero-, optionally cyclic C1-C18 hydrocarbyl, wherein R may form a ring by covalently attaching to the C1 or C5 carbon of the proximate pyrrolidinyl, or a pharmaceutically-acceptable salt thereof.

In embodiments:
R is methyl substituted with an optionally substituted, C3-C10 cyclic or heterocyclic group;
R is methyl substituted with an optionally substituted, C5, C6 or C10 aryl or heteoraryl group comprising 1, 2 or 3 N, O or S heteroatoms;
the compound comprises a structure of Table 1; and/or
the compound covalently reacts with Cys38 of the 14-3-3 protein and stabilizes or enhances interaction of a 14-3-3 protein with a substrate thereof.

In an aspect the invention provides a pharmaceutical composition comprising a compound herein and a pharmaceutically-acceptable excipient.

In embodiments the composition is in unit dosage or packaging.

The invention encompasses all combinations of the particular embodiments recited herein, as if each combination had been laboriously recited.

### DESCRIPTION OF PARTICULAR EMBODIMENTS OF THE INVENTION

Unless contraindicated or noted otherwise, in these descriptions and throughout this specification, the terms “a” and “an” mean one or more, the term “or” means and/or. It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein, including citations therein, are hereby incorporated by reference in their entirety for all purposes.

The term hydrocarbyl refers to hydrocarbon radical, including alkyl, akenyl, alkynyl and aryl.

The term “alkyl” refers to a hydrocarbon group selected from linear and branched saturated hydrocarbon groups of 1-18, or 1-12, or 1-6 carbon atoms. Examples of the alkyl group include methyl, ethyl,1-propyl or n-propyl (“n-Pr”), 2-propyl or isopropyl (“i-Pr”), 1-butyl or n-butyl (“n-Bu”), 2-methyl-1-propyl or isobutyl (“i-Bu”), 1-methylpropyl or s-butyl (“s-Bu”), and 1,1-dimethylethyl or t-butyl (“t-Bu”). Other examples of the alkyl group include 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl and 3,3-dimethyl-2-butyl groups.

Lower alkyl means 1-8, preferably 1-6, more preferably 1-4 carbon atoms; lower alkenyl or alkynyl means 2-8, 2-6 or 2-4 carbon atoms.

The term “alkenyl” refers to a hydrocarbon group selected from linear and branched hydrocarbon groups comprising at least one C═C double bond and of 2-18, or 2-12, or 2-6 carbon atoms. Examples of the alkenyl group may be selected from ethenyl or vinyl, prop-1-enyl, prop-2-enyl, 2-methylprop-1-enyl, but-1-enyl, but-2-enyl, but-3-enyl, buta-1,3-dienyl, 2-methylbuta-1,3-diene, hex-1-enyl, hex-2-enyl, hex-3-enyl, hex-4-enyl, and hexa-1,3-dienyl groups.

The term “alkynyl” refers to a hydrocarbon group selected from linear and branched hydrocarbon group, comprising at least one C═C triple bond and of 2-18, or 2-12, or 2-6 carbon atoms. Examples of the alkynyl group include ethynyl, 1-propynyl, 2-propynyl (propargyl), 1-butynyl, 2-butynyl, and 3-butynyl groups.

The term “cycloalkyl” refers to a hydrocarbon group selected from saturated and partially unsaturated cyclic hydrocarbon groups, comprising monocyclic and polycyclic (e.g., bicyclic and tricyclic) groups. For example, the cycloalkyl group may be of 3-12, or 3-8, or 3-6 carbon atoms. Even further for example, the cycloalkyl group may be a monocyclic group of 3-12, or 3-8, or 3-6 carbon atoms. Examples of the monocyclic cycloalkyl group include cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl, and cyclododecyl groups. Examples of the bicyclic cycloalkyl groups include those having 7-12 ring atoms arranged as a bicycle ring selected from [4,4], [4,5], [5,5], [5,6] and [6,6] ring systems, or as a bridged bicyclic ring selected from bicyclo[2.2.1]heptane, bicyclo[2.2.2]octane, and bicyclo[3.2.2]nonane. The ring may be saturated or have at least one double bond (i.e. partially unsaturated), but is not fully conjugated, and is not aromatic, as aromatic is defined herein.

The term “aryl” herein refers to a group selected from: 5- and 6-membered carbocyclic aromatic rings, for example, phenyl; bicyclic ring systems such as 7-12 membered bicyclic ring systems wherein at least one ring is carbocyclic and aromatic, selected, for example, from naphthalene, indane, and 1,2,3,4-tetrahydroquinoline; and tricyclic ring systems such as 10-15 membered tricyclic ring systems wherein at least one ring is carbocyclic and aromatic, for example, fluorene.

For example, the aryl group is selected from 5- and 6-membered carbocyclic aromatic rings fused to a 5- to 7-membered cycloalkyl or heterocyclic ring optionally comprising at least one heteroatom selected from N, O, and S, provided that the point of attachment is at the carbocyclic aromatic ring when the carbocyclic aromatic ring is fused with a heterocyclic ring, and the point of attachment can be at the carbocyclic aromatic ring or at the cycloalkyl group when the carbocyclic aromatic ring is fused with a cycloalkyl group. Bivalent radicals formed from substituted benzene derivatives and having the free valences at ring atoms are named as substituted phenylene radicals. Bivalent radicals derived from univalent polycyclic hydrocarbon radicals whose names end in “-yl” by removal of one hydrogen atom from the carbon atom with the free valence are named by adding “-idene” to the name of the corresponding univalent radical, e.g., a naphthyl group with two points of attachment is termed naphthylidene. Aryl, however, does not encompass or overlap with heteroaryl, separately defined below. Hence, if one or more carbocyclic aromatic rings are fused with a heterocyclic aromatic ring, the resulting ring system is heteroaryl, not aryl, as defined herein.

The term “halogen” or “halo” refers to F, Cl, Br or I.

The term “heteroalkyl” refers to alkyl comprising at least one heteroatom.

The term “heteroaryl” refers to a group selected from:

5- to 7-membered aromatic, monocyclic rings comprising 1, 2, 3 or 4 heteroatoms selected from N, O, and S, with the remaining ring atoms being carbon;

8- to 12-membered bicyclic rings comprising 1, 2, 3 or 4 heteroatoms, selected from N, O, and S, with the remaining ring atoms being carbon and wherein at least one ring is aromatic and at least one heteroatom is present in the aromatic ring; and

11- to 14-membered tricyclic rings comprising 1, 2, 3 or 4 heteroatoms, selected from N, O, and S, with the remaining ring atoms being carbon and wherein at least one ring is aromatic and at least one heteroatom is present in an aromatic ring.

For example, the heteroaryl group includes a 5- to 7-membered heterocyclic aromatic ring fused to a 5- to 7-membered cycloalkyl ring. For such fused, bicyclic heteroaryl ring systems wherein only one of the rings comprises at least one heteroatom, the point of attachment may be at the heteroaromatic ring or at the cycloalkyl ring.

When the total number of S and O atoms in the heteroaryl group exceeds 1, those heteroatoms are not adjacent to one another. In some embodiments, the total number of S and O atoms in the heteroaryl group is not more than 2. In some embodiments, the total number of S and O atoms in the aromatic heterocycle is not more than 1.

Examples of the heteroaryl group include, but are not limited to, (as numbered from the linkage position assigned priority 1) pyridyl (such as 2-pyridyl, 3-pyridyl, or 4-pyridyl), cinnolinyl, pyrazinyl, 2,4-pyrimidinyl, 3,5-pyrimidinyl, 2,4-imidazolyl, imidazopyridinyl, isoxazolyl, oxazolyl, thiazolyl, isothiazolyl, thiadiazolyl, tetrazolyl, thienyl, triazinyl, benzothienyl, furyl, benzofuryl, benzoimidazolyl, indolyl, isoindolyl, indolinyl, phthalazinyl, pyrazinyl, pyridazinyl, pyrrolyl, triazolyl, quinolinyl, isoquinolinyl, pyrazolyl, pyrrolopyridinyl (such as 1H-pyrrolo[2,3-b]pyridin-5-yl), pyrazolopyridinyl (such as 1H-pyrazolo[3,4-b]pyridin-5-yl), benzoxazolyl (such as benzo[d]oxazol-6-yl), pteridinyl, purinyl, 1-oxa-2,3-diazolyl, 1-oxa-2,4-diazolyl, 1-oxa-2,5-diazolyl, 1-oxa-3,4-diazolyl, 1-thia-2,3-diazolyl, 1-thia-2,4-diazolyl, 1-thia-2,5-diazolyl, 1-thia-3,4-diazolyl, furazanyl, benzofurazanyl, benzothiophenyl, benzothiazolyl, benzoxazolyl, quinazolinyl, quinoxalinyl, naphthyridinyl, furopyridinyl, benzothiazolyl (such as benzo[d]thiazol-6-yl), indazolyl (such as 1H-indazol-5-yl) and 5,6,7,8-tetrahydroisoquinoline.

The term “heterocyclic” or “heterocycle” or “heterocyclyl” refers to a ring selected from 4- to 12-membered monocyclic, bicyclic and tricyclic, saturated and partially unsaturated rings comprising at least one carbon atoms in addition to 1, 2, 3 or 4 heteroatoms, selected from oxygen, sulfur, and nitrogen. “Heterocycle” also refers to a 5- to 7-membered heterocyclic ring comprising at least one heteroatom selected from N, O, and S fused with 5-, 6-, and/or 7-membered cycloalkyl, carbocyclic aromatic or heteroaromatic ring, provided that the point of attachment is at the heterocyclic ring when the heterocyclic ring is fused with a carbocyclic aromatic or a heteroaromatic ring, and that the point of attachment can be at the cycloalkyl or heterocyclic ring when the heterocyclic ring is fused with cycloalkyl.

“Heterocycle” also refers to an aliphatic spirocyclic ring comprising at least one heteroatom selected from N, O, and S, provided that the point of attachment is at the heterocyclic ring. The rings may be saturated or have at least one double bond (i.e. partially unsaturated). The heterocycle may be substituted with oxo. The point of the attachment may be carbon or heteroatom in the heterocyclic ring. A heterocyle is not a heteroaryl as defined herein.

Examples of the heterocycle include, but not limited to, (as numbered from the linkage position assigned priority 1) 1-pyrrolidinyl, 2-pyrrolidinyl, 2,4-imidazolidinyl, 2,3-pyrazolidinyl, 1-piperidinyl, 2-piperidinyl, 3-piperidinyl, 4-piperidinyl, 2,5-piperazinyl, pyranyl, 2-morpholinyl, 3-morpholinyl, oxiranyl, aziridinyl, thiiranyl, azetidinyl, oxetanyl, thietanyl, 1,2-dithietanyl, 1,3-dithietanyl, dihydropyridinyl, tetrahydropyridinyl, thiomorpholinyl, thioxanyl, piperazinyl, homopiperazinyl, homopiperidinyl, azepanyl, oxepanyl, thiepanyl, 1,4-oxathianyl, 1,4-dioxepanyl, 1,4-oxathiepanyl, 1,4-oxaazepanyl, 1,4-dithiepanyl, 1,4-thiazepanyl and 1,4-diazepane 1,4-dithianyl, 1,4-azathianyl, oxazepinyl, diazepinyl, thiazepinyl, dihydrothienyl, dihydropyranyl, dihydrofuranyl, tetrahydrofuranyl, tetrahydrothienyl, tetrahydropyranyl, tetrahydrothiopyranyl, 1-pyrrolinyl, 2-pyrrolinyl, 3-pyrrolinyl, indolinyl, 2H-pyranyl, 4H-pyranyl, 1,4-dioxanyl, 1,3-dioxolanyl, pyrazolinyl, pyrazolidinyl, dithianyl, dithiolanyl, pyrazolidinylimidazolinyl, pyrimidinonyl, 1,1-dioxo-thiomorpholinyl, 3-azabicyco[3.1.0]hexanyl, 3-azabicyclo[4.1.0]heptanyl and azabicyclo[2.2.2]hexanyl. Substituted heterocycle also includes ring systems substituted with one or more oxo moieties, such as piperidinyl N-oxide, morpholinyl-N-oxide, 1-oxo-1-thiomorpholinyl and 1, 1-dioxo-1-thiomorpholinyl.

Substituents are selected from: halogen, —R′, —OR′, ═O, ═NR′, ═N—OR′, —NR′R″, —SR′, —SiR′R″R″′, —OC(O)R′, —C(O)R′, —CO₂R′, —CONR′R″, —OC(O)NR′R″, —NR″C(O)R′, —NR′—C(O)NR″R″′, —NR′—SO₂NR″″, —NR″CO₂R′, —NH—C(NH₂)═NH, —NR′C(NH₂)═NH, —NH—C(NH₂)═NR′, —S(O)R′, —SO₂R′, —SO₂NR′R″, —NR″SO₂R, —CN and —NO₂, —N₃, —CH(Ph)₂, perfluoro(C1-C4)alkoxy and perfluoro(C1-C4)alkyl, in a number ranging from zero to three, with those groups having zero, one or two substituents being particularly preferred. R, R′, R″ and R″′ each independently refer to hydrogen, unsubstituted (C1-C8)alkyl and heteroalkyl, unsubstituted aryl, aryl substituted with one to three halogens, unsubstituted alkyl, alkoxy or thioalkoxy groups, or aryl-(C1-C4)alkyl groups. When R′ and R″ are attached to the same nitrogen atom, they can be combined with the nitrogen atom to form a 5-, 6- or 7-membered ring. Hence, —NR′R″ includes 1-pyrrolidinyl and 4-morpholinyl, “alkyl” includes groups such as trihaloalkyl (e.g., —CF₃ and —CH₂CF₃), and when the aryl group is 1,2,3,4-tetrahydronaphthalene, it may be substituted with a substituted or unsubstituted (C3-C7)spirocycloalkyl group. The (C3-C7)spirocycloalkyl group may be substituted in the same manner as defined herein for “cycloalkyl”.

Preferred substituents are selected from: halogen, —R′, —OR′, ═O, —NR′R″, —SR′, —SiR′R″R″′, —OC(O)R′, —C(O)R′, —CO₂R′, —CONR′R″, —OC(O)NR′R″, —NR″C(O)R′, —NR″CO₂R′, —NR′—SO₂NR″R″′, —S(O)R′, —SO₂R′, —SO₂NR′R″, —NR″SO₂R, —CN and —NO₂, perfluoro(C1-C4)alkoxy and perfluoro(C1-C4)alkyl, where R′ and R″ are as defined above.

The term “fused ring” refers to a polycyclic ring system, e.g., a bicyclic or tricyclic ring system, in which two rings share only two ring atoms and one bond in common. Examples of fused rings may comprise a fused bicyclic cycloalkyl ring such as those having from 7 to 12 ring atoms arranged as a bicyclic ring selected from [4,4], [4,5], [5,5], [5,6] and [6,6] ring systems as mentioned above; a fused bicyclic aryl ring such as 7 to 12 membered bicyclic aryl ring systems as mentioned above, a fused tricyclic aryl ring such as 10 to 15 membered tricyclic aryl ring systems mentioned above; a fused bicyclic heteroaryl ring such as 8- to 12-membered bicyclic heteroaryl rings as mentioned above, a fused tricyclic heteroaryl ring such as 11- to 14-membered tricyclic heteroaryl rings as mentioned above; and a fused bicyclic or tricyclic heterocyclyl ring as mentioned above.

The compounds may contain an asymmetric center and may thus exist as enantiomers. Where the compounds possess two or more asymmetric centers, they may additionally exist as diastereomers. Enantiomers and diastereomers fall within the broader class of stereoisomers. All such possible stereoisomers as substantially pure resolved enantiomers, racemic mixtures thereof, as well as mixtures of diastereomers are intended to be included. All stereoisomers of the compounds and/or pharmaceutically acceptable salts thereof are intended to be included. Unless specifically mentioned otherwise, reference to one isomer applies to any of the possible isomers. Whenever the isomeric composition is unspecified, all possible isomers are included.

The compounds of the invention may also contain unnatural proportions of atomic isotopes at one or more of the atoms that constitute such compounds, such as deuterium, e.g. —CD₃, CD₂H or CDH₂ in place of methyl. For example, the compounds may be radiolabeled with radioactive isotopes, such as for example tritium (³H), iodine-125 (¹²⁵I) or carbon-14 (¹⁴C). All isotopic variations of the compounds of the invention, whether radioactive or not, are intended to be encompassed within the scope of the invention.

### Examples

We performed a fluorescence polarization screen with our cysteine-reactive covalent ligand libraries to identify 14-3-3 covalent molecular glues that enhance interactions of SFN with phosphopeptide 14-3-3 interacting sequences ERα. This screen was performed with recombinant pure 14-3-3 SFN protein and a 5-FAM- or rhodamine-conjugated ERα phosphopeptide substrate looking for cysteine-reactive covalent ligands that would stabilize 14-3-3 interactions with the phosphopeptide substrate. We identified compound EN171 that stabilized 14-3-3 interactions with the ERα phosphopeptide substrate to the same extent as our positive control compound Fusicoccin A. EN171 was able to significantly enhance SFN interactions not only with ERα phosphopeptide substrates, but also with those from YAP and TAZ transcription factors in a dose-responsive manner with EC50s of 9, 45, and 107 mM, respectively. This enhanced interaction of 14-3-3 with the estrogen receptor and TAZ phosphopeptide substrates were attenuated with the 14-3-3 inhibitor R18. Using LC-MS/MS to analyze EN171 modified tryptic peptides from pure SFN, we demonstrate that EN171 reacts with C38 and C96 of SF. To further demonstrate EN171 interactions with SFN, we synthesized an alkyne-functionalized probe of EN171, EN171-alkyne, and showed that this probe covalently modified pure SFN protein in a dose-responsive manner by labeling SFN with this probe, followed by appendage of rhodamine-azide by copper-catalyzed azide-alkyne cycloaddition (CuAAC), separating protein by SDS/PAGE and visualizing in-gel fluorescence. We further show that mutation of both C38 and C96 on SFN to serines led to no labeling of SFN by the EN171-alkyne probe. We further show that mutation of C38 and C96 of SFN to serines also significantly attenuated EN171-mediated stabilization of 14-3-3 interactions with ERα and TAZ. Consistent with EN171 sequestration of ERα and Hippo pathway transcription factors YAP and TAZ out of the nucleus and in the cytosol in cancer cells, we observed dose-responsive inhibition of ERα and Hippo pathway transcription factor luciferase reporter activity in T47D and MCF7 breast cancer cells, respectively. To demonstrate that this cellular inhibitory activity was due to SFN, we demonstrated that SFN knockdown significantly attenuated EN171-mediated inhibition of ERα and Hippo pathway transcriptional activity in breast cancer cells.

We preformed extensive SAR analysis confirming activity of diverse analogues of EN-171, including compounds of Table 1, and representative compounds of general formula I (supra); we demonstrated that each of these analogues covalently reacts with Cys38 of the 14-3-3 protein and stabilizes or enhances interaction of the 14-3-3 protein with the substrate.

TABLE 1

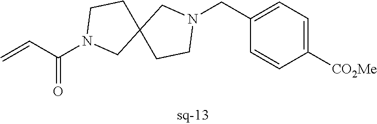 [SMILES: C=CC(=O)N1CCC2(CCN(Cc3ccc(C(=O)OC)cc3)C2)C1]

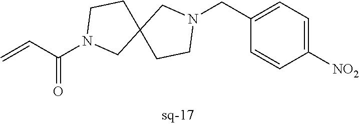 [SMILES: C=CC(=O)N1CCC2(CCN(Cc3ccc([N+](=O)[O-])cc3)C2)C1]

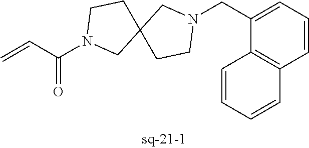 [SMILES: C=CC(=O)N1CCC2(CCN(Cc3cccc4ccccc34)C2)C1]

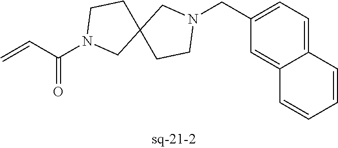 [SMILES: C=CC(=O)N1CCC2(CCN(Cc3ccc4ccccc4c3)C2)C1]

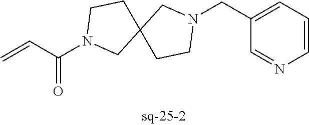 [SMILES: C=CC(=O)N1CCC2(CCN(Cc3cccnc3)C2)C1]

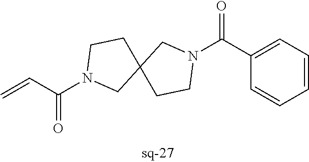 [SMILES: C=CC(=O)N1CCC2(CCN(C(=O)c3ccccc3)C2)C1]

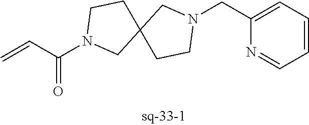 [SMILES: C=CC(=O)N1CCC2(CCN(Cc3ccccn3)C2)C1]

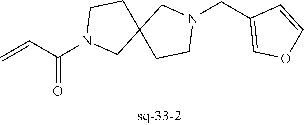 [SMILES: C=CC(=O)N1CCC2(CCN(Cc3ccoc3)C2)C1]

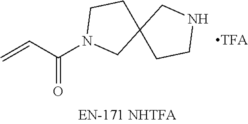

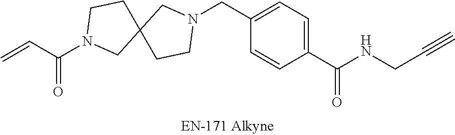 [SMILES: C#CCNC(=O)c1ccc(CN2CCC3(CCN(C(=O)C=C)C3)C2)cc1]

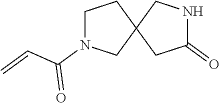

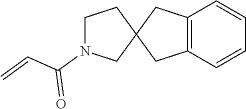 [SMILES: C=CC(=O)N1CCC2(Cc3ccccc3C2)C1]

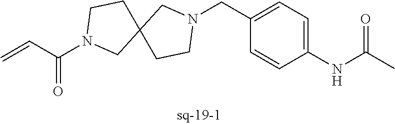 [SMILES: C=CC(=O)N1CCC2(CCN(Cc3ccc(NC(C)=O)cc3)C2)C1]

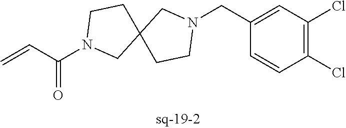 [SMILES: C=CC(=O)N1CCC2(CCN(Cc3ccc(Cl)c(Cl)c3)C2)C1]

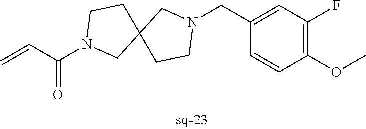 [SMILES: C=CC(=O)N1CCC2(CCN(Cc3ccc(OC)c(F)c3)C2)C1]

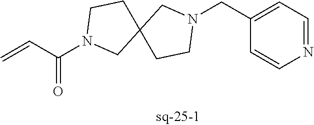 [SMILES: C=CC(=O)N1CCC2(CCN(Cc3ccncc3)C2)C1]

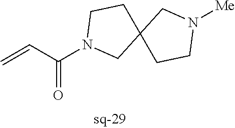

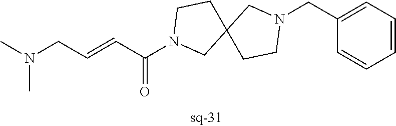 [SMILES: CN(C)C/C=C/C(=O)N1CCC2(CCN(Cc3ccccc3)C2)C1]

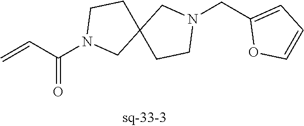 [SMILES: C=CC(=O)N1CCC2(CCN(Cc3ccco3)C2)C1]

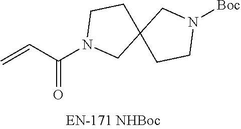 [SMILES: C=CC(=O)N1CCC2(CCN(C(=O)OC(C)(C)C)C2)C1]

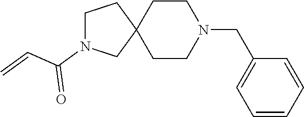 [SMILES: C=CC(=O)N1CCC2(CCN(Cc3ccccc3)CC2)C1]

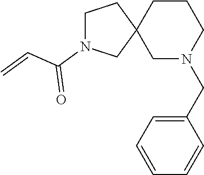 [SMILES: C=CC(=O)N1CCC2(CCCN(Cc3ccccc3)C2)C1]

### REFERENCES

(1) Spradlin, J. N.; Zhang, E.; Nomura, D. K. Reimagining Druggability Using Chemoproteomic Platforms. Acc. Chem. Res. 2021, 54 (7), 1801-1813. https://doi.org/10.1021/acs.accounts.1c00065.
(2) Burslem, G. M.; Crews, C. M. Proteolysis-Targeting Chimeras as Therapeutics and Tools for Biological Discovery. Cell 2020, 181 (1), 102-114. https://doi.org/10.1016/j.cell.2019.11.031.
(3) Schreiber, S. L. Chemistry and Biology of the Immunophilins and Their Immunosuppressive Ligands. Science 1991, 251 (4991), 283-287. https://doi.org/10.1126/science.1702904.
(4) Schreiber, S. L. The Rise of Molecular Glues. Cell 2021, 184 (1), 3-9. https://doi.org/10.1016/j.cell.2020.12.020.
(5) Lu, G.; Middleton, R. E.; Sun, H.; Naniong, M.; Ott, C. J.; Mitsiades, C. S.; Wong, K.-K.; Bradner, J. E.; Kaelin, W. G. The Myeloma Drug Lenalidomide Promotes the Cereblon-Dependent Destruction of Ikaros Proteins. Science 2014, 343 (6168), 305-309. https://doi.org/10.1126/science.1244917.
(6) Milroy, L.-G.; Grossmann, T. N.; Hennig, S.; Brunsveld, L.; Ottmann, C. Modulators of Protein-Protein Interactions. Chem. Rev. 2014, 114 (9), 4695-4748. https://doi.org/10.1021/cr400698c.
(7) Ward, C. C.; Kleinman, J. I.; Brittain, S. M.; Lee, P. S.; Chung, C. Y. S.; Kim, K.; Petri, Y.; Thomas, J. R.; Tallarico, J. A.; McKenna, J. M.; Schirle, M.; Nomura, D. K. Covalent Ligand Screening Uncovers a RNF4 E3 Ligase Recruiter for Targeted Protein Degradation Applications. ACS Chem. Biol. 2019, 14 (11), 2430-2440. https://doi.org/10.1021/acschembio.8b01083.
(8) Sijbesma, E.; Hallenbeck, K. K.; Leysen, S.; de Vink, P. J.; Skóra, L.; Jahnke, W.; Brunsveld, L.; Arkin, M. R.; Ottmann, C. Site-Directed Fragment-Based Screening for the Discovery of Protein-Protein Interaction Stabilizers. J. Am. Chem. Soc. 2019, 141 (8), 3524-3531. https://doi.org/10.1021/jacs.8b11658.
(9) Spradlin, J. N.; Hu, X.; Ward, C. C.; Brittain, S. M.; Jones, M. D.; Ou, L.; To, M.; Proudfoot, A.; Ornelas, E.; Woldegiorgis, M.; Olzmann, J. A.; Bussiere, D. E.; Thomas, J. R.; Tallarico, J. A.; McKenna, J. M.; Schirle, M.; Maimone, T. J.; Nomura, D. K. Harnessing the Anti-Cancer Natural Product Nimbolide for Targeted Protein Degradation. Nat. Chem. Biol. 2019, 15 (7), 747-755. https://doi.org/10.1038/s41589-019-0304-8.
(10) Boike, L.; Cioffi, A. G.; Majewski, F. C.; Co, J.; Henning, N. J.; Jones, M. D.; Liu, G.; McKenna, J. M.; Tallarico, J. A.; Schirle, M.; Nomura, D. K. Discovery of a Functional Covalent Ligand Targeting an Intrinsically Disordered Cysteine within MYC. Cell Chem. Biol. 2021, 28 (1), 4-13.e17. https://doi.org/10.1016/j.chembiol.2020.09.001.

1. A method to identify a molecular glue, comprising measuring fluorescence polarization of a 14-3-3 protein and a fluorescently-labeled phosphopeptide substrate thereof comprising transcription factor 14-3-3 interacting sequences, in the presence of candidate cysteine-reactive covalent ligands to identify a ligand that covalently reacts with Cys38 of the 14-3-3 protein and stabilizes or enhances interaction of the 14-3-3 protein with the substrate.

2. The method of claim 1, wherein the transcription factor is an oncogenic transcription factor such as YAP and TAZ.

3. The method of claim 1, wherein the transcription factor is ERα.

4. The method of claim 1, wherein the label is 5-FAM or rhodamine.

5. The method of claim 1, wherein the 14-3-3 protein is wild-type (not engineered).

6. The method of claim 1, wherein the 14-3-3 protein is stratifin (SFN).

7. The method of claim 1, wherein the ligands or ligand has an n-octanol-water partition coefficient, K_{ow} (log P) in −0.4 to +5.6 range.

8. The method of claim 1, wherein the ligands or ligand complies with Lipinski's rule of five for drug-likeness, and/or with Ghose's filter:
(a) partition coefficient log P in −0.4 to +5.6 range;
(b) molar refractivity from 40 to 130;
(c) molecular weight from 180 to 480; and
(d) number of atoms from 20 to 70 (includes H-bond donors [e.g. OHs and NHs] and H-bond acceptors [e.g. Ns and Os]).

9. A drug discovery platform configured for the method of claim 1.

10. A compound comprising a 7-substituted, 2,7-diazaspiro[4.4]nonane-2-acrylamide, or a pharmaceutically-acceptable salt thereof.

11. The compound of claim 10, that is 7-substituted with R, wherein:
a) R is selected from optionally substituted heteroatom and optionally substituted, optionally hetero-, optionally cyclic C1-C18 hydrocarbyl, wherein R may form a ring by covalently attaching to the C1 or C5 carbon of the proximate pyrrolidinyl;
b) R is methyl substituted with an optionally substituted, C3-C10 cyclic or heterocyclic group; or
c) R is methyl substituted with an optionally substituted, C5, C6 or C10 aryl or heteroaryl group comprising 1, 2 or 3 N, O or S heteroatoms.

12. The compound of claim 10, comprising a structure of Table 1.

13. The compound of claim 10, that covalently reacts with Cys38 of the 14-3-3 protein and stabilizes or enhances interaction of a 14-3-3 protein with a substrate thereof.

14. A compound of general formula I: 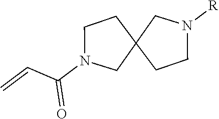
wherein R is selected from optionally substituted heteroatom and optionally substituted, optionally hetero-, optionally cyclic C1-C18 hydrocarbyl, wherein R may form a ring by covalently attaching to the C1 or C5 carbon of the proximate pyrrolidinyl,
or a pharmaceutically-acceptable salt thereof.

15. The compound of claim 14, wherein R is methyl substituted with an optionally substituted, C3-C10 cyclic or heterocyclic group.

16. The compound of claim 14, wherein is methyl substituted with an optionally substituted, C5, C6 or C10 aryl or heteroaryl group comprising 1, 2 or 3 N, O or S heteroatoms.

17. The compound of claim 14, comprising a structure of Table 1.

18. The compound of claim 14, that covalently reacts with Cys38 of the 14-3-3 protein and stabilizes or enhances interaction of a 14-3-3 protein with a substrate thereof.

19. A pharmaceutical composition comprising a compound of claim 10, and a pharmaceutically-acceptable excipient, preferably in unit dosage or packaging.

20. A pharmaceutical composition comprising a compound of claim 14, and a pharmaceutically-acceptable excipient, preferably in unit dosage or packaging.
